(19) United States
(12) Patent Application Publication
(10) Pub. No.: US 2004/0249384 A1
(43) Pub. Date: Dec. 9, 2004

(54) Compacting broach

(76) Inventors: J. David Blaha, Ann Arbor, MI (US); Angelo DeLollis, Milan (IT); Irina Timmerman, Bartlett, TN (US); Henry Faber, Memphis, TN (US)

Correspondence Address: WRIGHT MEDICAL TECHNOLOGY, INC., 5677 AIRLINE ROAD, ARLINGTON, TN, 38002-9501, US

(21) Appl. No.: 10/764,726

(22) Filed: Jan. 26, 2004

Related U.S. Application Data
(60) Provisional application No. 60/444,631, filed on Feb. 4, 2003.

(51) Int. Cl. A61B 17/32; A61F 2/46

(52) U.S. Cl. 606/85; 623/22.12

(57) ABSTRACT

A compacting broach comprises an elongate body and a distal tip at a forward end of the body. The body plurality of corner edges corresponding to the corner edges of a prosthetic component, cutting teeth along the corner edges and side walls between the cutting teeth of adjacent corner edges. The side walls extend inwardly from the cutting teeth of adjacent corner edges and compact cancellous bone as the cutting teeth penetrate cortical bone during advancement of the broach in the intramedullary canal. The distal tip comprises a plurality of fins radial to a central longitudinal axis of the broach and extending longitudinally, distally from the body. The fins contact the side of the canal at locations about a central axis of the canal to center the broach in the canal prior to the cutting teeth engaging cortical bone as the broach is advanced therein.

### RELATED APPLICATIONS

This application claims priority to U.S. Provisional Patent Application Ser. No. 60/444,631, filed Feb. 4, 2003.

### FIELD OF THE INVENTION

The present invention relates generally to the formation of cavities in long bones to receive prosthetic components and, more particularly, to compacting broaches which form cavities in femoral shafts by compacting the bone material.

### BACKGROUND OF THE INVENTION

Prosthetic components implanted in long bones oftentimes comprise elongate stems or shanks anchored or fixated in cavities formed in the bones. Prosthetic femoral components used in hip procedures typically comprise a stem connected to a prosthetic neck which in turn is connected to a prosthetic head that articulates with the corresponding acetabular portion of the hip joint. The stems are ordinarily fixated in cavities prepared in the upper end of the femoral shaft, and various approaches have been proposed for fixation of the stems including cemented fixation, mechanical fixation such as is accomplished using bolts inserted transversely through the femoral shaft and stem of the prosthetic femoral component, “square peg” fixation involving the installation of a generally rectangular cross-section stem in a substantially circular cross-section cavity as represented by the Resolution®, Zweymuller® and Profemur™ Z fixations, and “anatomical” fixation as represented by the PERFECTA® stems of Wright Medical Technology.

Many prior approaches to fixation of femoral component stems in cavities formed in the femoral shaft have associated disadvantages. For example, one disadvantage of “square peg” fixation is the introduction of stress-risers in the bone at the corners of the stem cross-section, thereby risking splitting of the bone at the corners where the bone is most vulnerable to high stress concentrations. Another disadvantage of “square peg” fixation is inadequate contact between flat sides of the stems and the wall of the cavity such that the interference fit of the stem with the bone is not maximized.

Rasps and broaches have been used to form cavities in femoral shafts to receive the stems of prosthetic femoral components. Most typically, the rasps and broaches have cutting teeth along all sides thereof to remove bone and thereby create the cavities. U.S. Pat. No. 6,488,714 B2 to Keller discloses a rasp having two toothed sections separated by smooth surfaces, and U.S. Pat. No. 3,815,599 to Deyerle discloses a rasp having cutting teeth along one side thereof. U.S. Pat. No. 4,671,275 to Deyerle discloses a rasp for cutting through the bone only at the corners of the rasp in order to preserve cancellous bone for compression against the irregular outer surface of the stem of the femoral component. In the Keller patent, the toothing is limited to the proximal length section of the rasp with a substantial distal length section of the rasp being smooth. In the Deyerle patents, the cutting or working sections of the rasps terminate at flat distal end surfaces which lead the rasps into the bone. The distal end configurations of prior rasps and broaches fail to provide any structure for centering the rasps and broaches in the intramedullary canals of the bones prior to the cutting teeth engaging cortical bone. As such, prior rasps and broaches may be introduced into the intramedullary canal off-center, such that the cutting teeth of the rasps and broaches do not evenly engage cortical bone as the rasps and broaches are advanced into the canals.

### SUMMARY OF THE INVENTION

Accordingly, it is an object of the present invention to center a compacting broach in the intramedullary canal of a bone prior to cutting teeth of the broach engaging cortical bone as the broach is advanced in the canal.

Another object of the present invention is to ensure even engagement of the cutting teeth of a compacting broach with cortical bone as the broach is advanced into the intramedullary canal of the bone.

It is also an object of the present invention is to improve the interference fit between a bone arid a prosthetic component installed in a cavity formed in the bone.

The present invention has as another object to reduce the risk of splitting a bone at the corners of a prosthetic component fixated in a cavity formed in the bone.

An additional object of the present invention is to facilitate proper penetration of a compacting broach into a bone to form a cavity therein for receiving a prosthetic component.

It is also an object of the present invention to create an environment promoting on-growth and in-growth of bone onto a prosthetic component fixated in a cavity formed in the bone.

These and other objects, advantages and benefits are realized with the present invention as generally characterized in a compacting broach for forming an elongate cavity in the intramedullary canal of a bone to receive a prosthetic component. The compacting broach is particularly designed to form a cavity in the intramedullary canal of the femoral shaft to receive the stem of a prosthetic femoral component in hip procedures. The compacting broach comprises an elongate body having a central longitudinal axis, a rearward end and a forward end, and a distal tip at the forward end of the body. The body has a plurality of corner edges extending longitudinally along the body to the distal tip and cutting teeth along the corner edges. The body has side walls connecting the cutting teeth of adjacent corner edges, and the side walls extend inwardly from the cutting teeth in the direction of the central longitudinal axis. In one embodiment, the side walls have a concave configuration. In another embodiment, each side wall is formed of first and second side wall segments extending angularly inwardly from the cutting teeth of the adjacent corner edges, respectively, to converge at a point. The corner edges of the body are matched to corresponding corner edges of the prosthetic component. The distal tip comprises a plurality of fins extending radial to the central longitudinal axis and extending longitudinally, distally from the forward end of the body to converge at an apex aligned with the central longitudinal axis. The fins have outer edge surfaces disposed inwardly of the cutting teeth and, as the compacting broach is advanced into the intramedullary canal, the outer surfaces of the fins contact the cortical bone without penetration thereof. The fins contact the cortical bone at locations about a longitudinal axis of the canal and center the compacting broach within the canal prior to the cutting teeth engaging the cortical bone. In one embodiment, the distal tip includes four fins at 90 degree spaced locations about the central longitudinal axis of the body to engage the cortical bone anteriorly, posteriorly, medially and laterally. In another embodiment, the distal tip comprises three fins arranged so as to define the corners of a triangle.

The present invention is also generally characterized in a method of forming an elongate cavity in the intramedullary canal of a bone to receive a prosthetic component and comprising the steps of introducing a distal tip of a compacting broach into the intramedullary canal, contacting the cortical bone with fins of the distal tip at locations about a longitudinal axis of the canal to center the compacting broach in the canal, advancing the compacting broach into the canal with the distal tip centering the compacting broach as it is advanced, cutting the cortical bone, as the compacting broach is advanced, with cutting teeth at corner edges of the compacting broach extending proximally from the distal tip, compacting cancellous bone, as the compacting broach is advanced in the canal, with side walls of the compacting broach extending inwardly between the cutting teeth of adjacent corner edges, withdrawing the compacting broach from the canal after advancement to a desired depth to leave an elongate cavity in the canal having a cross-sectional configuration defining a plurality of corners corresponding to the corner edges. After the compacting broach is withdrawn, a prosthetic component may be inserted in the cavity with corners of the prosthetic component aligned with the corners of the cavity without a press-fit. As the prosthetic component is inserted and advanced in the cavity, side walls of the prosthetic component further compact cancellous bone in the canal.

### BRIEF DESCRIPTION OF THE DRAWINGS

FIG. 1 is a perspective view of a compacting broach according to the present invention.

FIG. 2 is a sectional view of a body of the compacting broach.

FIG. 3 is a broken side view of a distal portion of the compacting broach illustrating the distal tip thereof.

FIG. 4 is a sectional view taken along line A-A of FIG. 3 showing the fins of the distal tip.

FIG. 5 is a sectional view showing an alternative arrangement for the fins.

FIG. 6 is a perspective view of an alternative compacting broach according to the present invention.

FIG. 7 is a sectional view of the body of the alternative compacting broach.

FIG. 8 is a sectional view of the femur depicting a cavity formed therein using the compacting broach of FIG. 1.

### DETAILED DESCRIPTION OF THE DRAWINGS

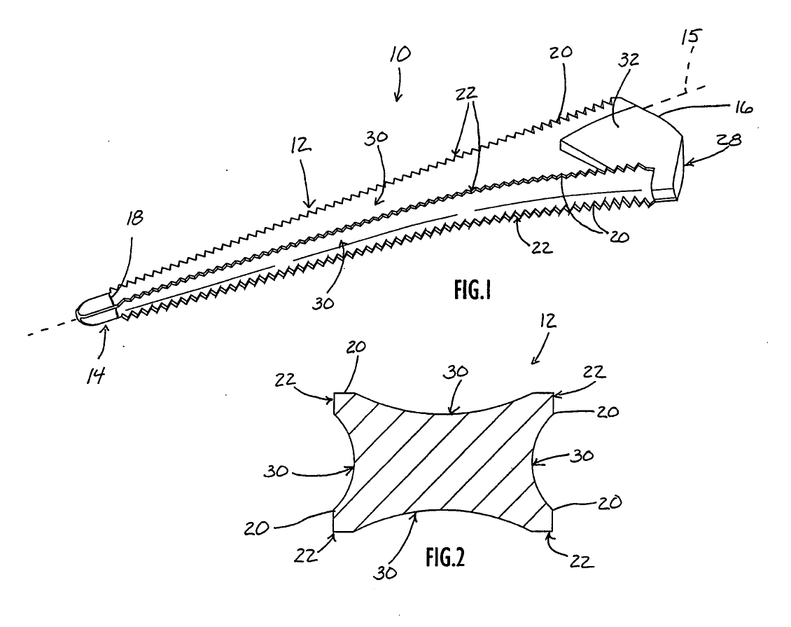

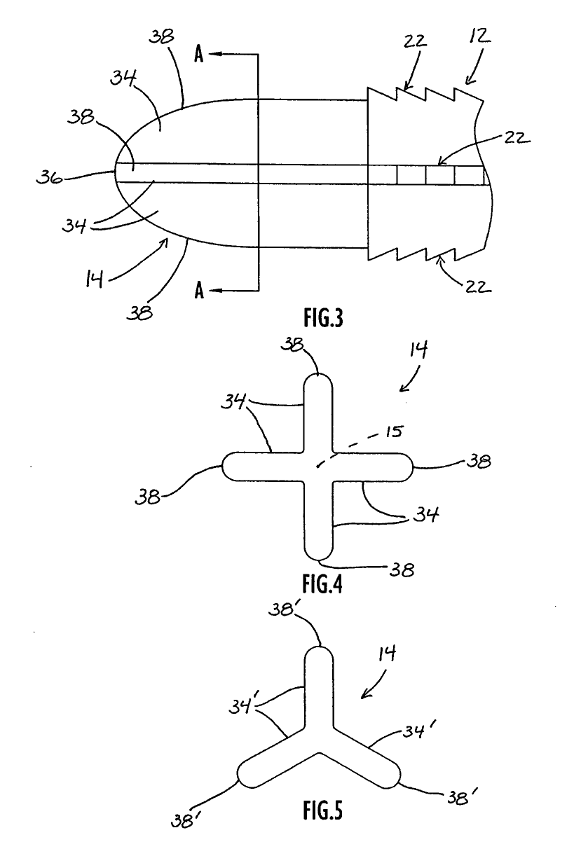

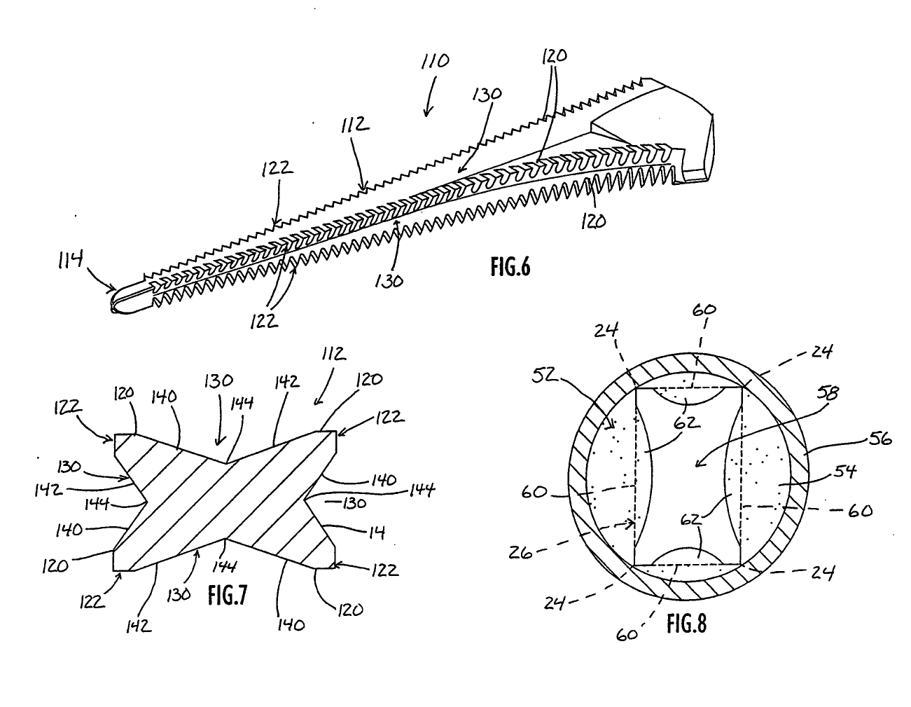

An exemplary compacting broach 10 according to the present invention is illustrated in FIG. 1 and comprises an elongate broach body 12 and a distal tip 14 at a forward end of body 12. Body 12 has a central longitudinal axis 15, a rearward end 16 defining the proximal end of broach 10 and a forward end 18 joined to distal tip 14. The distal tip 14 may be formed integrally, unitarily with body 12 or may be formed separately from body 12 and connected thereto in any suitable manner. Body 12 has a plurality of longitudinally extending corner edges 20 and cutting teeth 22 along the corner edges 20 as shown in FIGS. 1-3. The compacting broach 10 is designed to form a cavity in the femoral shaft for receiving the stem of a prosthetic femoral component implanted during a hip procedure, and the corner edges 20 of body 12 correspond in location, configuration and arrangement to corner edges 24 of the stem 26 shown in dotted lines in FIG. 8. Body 12 has four corner edges 20 which, if connected by straight lines, define a rectangular configuration in cross-section corresponding to the rectangular cross-sectional configuration of stem 26 as shown by FIGS. 2 and 8. The corner edges 20 extend distally along the body 12 from a location at or near the proximal end 16 to the distal tip 14, and the teeth 22 extend without interruption along the corner edges. The corner edges 20 follow the proximal to distal configuration of the stem 26, with the pair of corner edges 20 on one side of the central longitudinal axis 15 being straight from proximal to distal and the pair of corner edges on the opposite side of the central longitudinal axis 15 being at least partly curved from proximal to distal. Accordingly, the cross-section of the body 12 is non-uniform or variable from proximal to distal. The teeth 22 are designed to contact and penetrate through cortical bone when the compacting broach 10 is advanced into the femoral shaft in a longitudinal direction as explained further below. The proximal end 16 may comprise a connecting section 28 oriented at an angle to the longitudinal axis 15 for connection with a suitable handle.

The body 12 has smooth side walls 30 connecting the teeth 22 of adjacent corner edges 20, and the side walls 30 have a concave configuration between the teeth of adjacent corner edges. The side walls of the body 12 may be built up near the proximal end 16 thereof as shown in FIG. 1 by planar parallel wall segments 32 on opposite sides of the body 12 at the proximal end, only one wall segment 32 being visible in FIG. 1. The wall segments 32 may form part of the connecting section 28 at the proximal end of the broach and may provide structure facilitating adaptation of the proximal end for releasable connection with a handle.

As shown in FIGS. 1, 3 and 4, the distal tip 14 comprises a plurality of fins 34 extending longitudinally, distally from the forward end 18 of body 12 to converge at an apex aligned with the central longitudinal axis 15 and defining a distal end 36 for broach 10. The fins 34 are planar and extend radial to the central longitudinal axis 15, and four fins 34 may be provided at 90 degree spaced locations about the central longitudinal axis 15 to define a cross-shaped configuration in cross-section. The fins 34 have outer edge surfaces 38, respectively, which curve from the body 12 to the distal end 36, thereby providing the distal tip 14 with a longitudinally tapered or converging bullet-shaped configuration facilitating advancement of the compacting broach 10 in the intramedullary canal of the femur. Since the distal tip 14 is longitudinally tapered, the cross-sectional configuration of the distal tip is non-uniform or tapered from the body 12 to the distal end or apex 36. In the case of compacting broach 10, each fin 34 is aligned with a corner edge 20.

An alternative arrangement for the fins is illustrated in FIG. 5 which depicts distal tip 14 with three fins 34′ at radially spaced locations in which the outer edge surfaces 38′ of the fins 34′ define three corners of a triangle in cross-section. The fins 34′ can be equally spaced or non-equally spaced about the central longitudinal axis 15.

FIG. 2 depicts an alternative compacting broach 110 comprising body 112 and distal tip 114. Compacting broach 110 is similar to compacting broach 10 except that each side wall 130 for body 112 comprises a pair of side wall segments 140 and 142 angled inwardly toward one another from the cutting teeth 122 of adjacent corner edges 120 to converge at a point 144 between the adjacent corner edges. Using pairs of inwardly angled side wall segments between the cutting teeth of adjacent corner edges maximizes the amount of bone material that is compacted by the stem 26 of the prosthetic femoral component during insertion of the stem into a cavity formed in the femur using the compacting broach as explained further below.

Use of the compacting broach 10 may best be understood with reference to FIG. 8 which depicts a femoral shaft 50 comprising intramedullary canal 52 containing cancellous bone 54 surrounded by cortical bone 56. The compacting broach 10 is introduced in the intramedullary canal 52 of the femoral shaft 50 with the distal tip 14 leading the compacting broach as it is inserted. The fins 34 of the distal tip 14 center the compacting broach in the canal 52 prior to the cutting teeth 22 engaging cortical bone 56. The fins 34 contact the sides of the canal, defined by cortical bone 56, at locations about the central axis of the canal such that the compacting broach 10 is properly centered in the canal, but do not penetrate or cut the cortical bone 56. In the case of distal tip 14, the fins 34 may contact the sides of the canal anteriorly, posteriorly, medially and laterally, respectively. As the compacting broach 10 is further advanced in the canal, the cutting teeth 22, which protrude beyond the fins 34 in the radial direction, engage the cortical bone 56 and penetrate or cut the cortical bone at the corner edges 20. Even or uniform tooth engagement is ensured at all corner edges of the broach since the broach is centered in the canal. The side walls 30 of the body 12 do not cut the bone material but, rather, act by compacting the cancellous bone 54. The amount of compaction is variable due to the cross-shaped cross-sectional configuration of the body 12. The compacting broach 10 is inserted in the femoral shaft 50 to the proper distance and then withdrawn, leaving an elongate cavity 58 in the femoral shaft. As shown in FIG. 8, the cavity 58 has a cross-sectional configuration corresponding to the cross-section of body 12 with corners of the cavity 58 in cortical bone 56 corresponding to the corners 24 of the stem 26. When the stem 26 is introduced in the cavity 58 for implantation therein, the corners 24 of the stem 26 fit within the corners of cavity 58 without a press-fit, thusly eliminating stress concentrations in the cortical bone 56 at the corners of the stem. As the stem 26 is introduced, its side walls 60 shown in dotted lines in FIG. 8 compact and displace areas 62 of cancellous bone 54, thereby further compacting the bone material. An environment is thusly created in the femoral shaft that promotes bone on-growth or in-growth with the stem 26, which typically has an irregular or porous outer surface of titanium or other suitable material. Use of compacting broach 110 is similar to that described for compacting broach 10; however, the side wall segments 140 and 142 may provide a greater amount of bone material in areas 62 to be compacted by the stem 26.

What is claimed is:

1. A compacting broach for forming a cavity in the intramedullary canal of a bone to receive a prosthetic component, comprising
an elongate body having a central longitudinal axis, a rearward end, a forward end, and a plurality of cutting teeth on a surface of said elongated body and being matched to corresponding corners of the prosthetic component; and
a distal tip at said forward end of said elongate body, said distal tip comprising a plurality of members extending radially to said central longitudinal axis and extending longitudinally, distally from said forward end of said body to converge at an apex aligned with said central longitudinal axis, said fins being disposed inwardly of said cutting teeth.

2. A compacting broach for forming a cavity in the intramedullary canal of a bone to receive a prosthetic component, comprising
an elongate body having a central longitudinal axis, a rearward end, a forward end, a plurality of corner edges extending longitudinally along said body to said forward end, cutting teeth along said corner edges, and side walls connecting said cutting teeth of adjacent corner edges, said side walls extending inwardly from said cutting teeth in the direction of said central longitudinal axis, said corner edges being matched to corresponding corners of the prosthetic component; and
a distal tip at said forward end of said body, said distal tip comprising a plurality of fins extending radial to said central longitudinal axis and extending longitudinally, distally from said forward end of said body to converge at an apex aligned with said central longitudinal axis, said fins being disposed inwardly of said cutting teeth.

3. The compacting broach recited in claim 2 wherein said body comprises four corner edges matched to corresponding corners of a prosthetic component of rectangular cross-section.

4. The compacting broach recited in claim 2 wherein said side walls have a concave configuration.

5. The compacting broach recited in claim 2 wherein each of said side walls comprises first and second side wall segments extending angularly inwardly from said teeth of said adjacent corner edges, respectively, in the direction of said central longitudinal axis and converging at a point.

6. The compacting broach recited in claim 2 wherein said distal tip comprises four fins at 90 degree spaced locations about said central longitudinal axis.

7. The compacting broach recited in claim 6 wherein said fins are aligned with said teeth of said corner edges, respectively.

8. The compacting broach recited in claim 2 wherein said distal tip comprises three fins.

9. The compacting broach recited in claim 2 wherein said fins are planar.

10. The compacting broach recited in claim 2 wherein said distal tip has a bullet-shape in longitudinal profile.

11. A method of forming an elongate cavity in the intramedullary canal of a bone to receive a prosthetic component, comprising the steps of
introducing a distal tip of a compacting broach into the intramedullary canal;
contacting the cortical bone with fins of the distal tip at locations about a longitudinal axis of the canal to center the compacting broach in the canal;
advancing the compacting broach into the canal with the distal tip centering the compacting broach in the canal as it is advanced;
cutting, as the compacting broach is advanced, the cortical bone with cutting teeth disposed at corner edges of the compacting broach extending proximally from the distal tip;
compacting, as the compacting broach is advanced, cancellous bone in the canal with side walls of the compacting broach extending inwardly between the cutting teeth of adjacent corner edges;
withdrawing the compacting broach from the canal after advancement to a desired depth to leave an elongate cavity in the canal having a cross-sectional configuration defining a plurality of corners corresponding to the corner edges.

12. The method recited in claim 11 and further including, subsequent to said step of withdrawing, the steps of inserting a prosthetic component in the cavity with corners of the prosthetic component aligned with the corners of the cavity and, as the prosthetic component is inserted, compacting cancellous bone in the canal with side walls of the prosthetic component.
